(19) United States
(12) Patent Application Publication
(10) Pub. No.: US 2023/0210510 A1
(43) Pub. Date: Jul. 6, 2023

(54) System and methods to hemostatically close arterial and venous access locations

(71) Applicants: Stephens Robin, Burgess Hill (GB); Cunniffe Brendan, Galway (GB)

(72) Inventors: Stephens Robin, Burgess Hill (GB); Cunniffe Brendan, Galway (GB)

(21) Appl. No.: 18/008,514

(22) PCT Filed: Jun. 7, 2021
(86) PCT No.: PCT/GB2021/051403

§ 371 Date: Dec. 6, 2022

Related U.S. Application Data
(60) Provisional application No. 63/035,848, filed on Jun. 8, 2020.

(51) Int. Cl. A61B 17/00

(52) U.S. Cl. CPC A61B 17/0057; A61B 2017/00004; A61B 2017/00557; A61B 2017/00623

(57) ABSTRACT

The present invention relates generally to the field of closing of openings in vessel walls, i.e. closure of vascular holes, in particular access to and closure of an access point through the wall of a human vessel, and more specifically to the hemostatic closure and structural support of an opening such as an access point through a fluid filled vessel, particularly a human vessel, be that an artery, vein or other body conduit.

### FIELD OF THE INVENTION

The present invention relates generally to the field of closing of openings in vessel walls, i.e. closure of vascular holes, in particular access to and closure of an access point through the wall of a human vessel, and more specifically to the hemostatic closure and structural support of an opening such as an access point through a fluid filled vessel, particularly a human vessel, be that an artery, vein or other body conduit.

### BACKGROUND

Transcatheter endovascular procedures for the therapeutic treatment of Aortic Valve disease (TAVR), Mitral Valve disease (MVR), Abdominal (AAA) and Thoracic Aneurysm (TAA), Tricuspid Valve deficiency (TVR), etc. have become commonplace in cardiac surgery. In order to gain access to the specific region of the heart requiring treatment, the transfemoral route is by far the most commonly used access location. With this approach, a percutaneous incision is made into the Common Femoral Artery (Femoral Vein for Right-Side Heart procedures) in the region of the patient’s groin and a large bore Introducer sheath (typically in the range of 12Fr to 24Fr; FR = French units - a measurement of the outer diameter of a catheter; 1 Fr = 0.33 mm) is used to introduce the therapeutic device into the central vasculature. Post procedural bleeding and vascular complications associated with this access point have been identified as a major concern in catheter-based cardiac interventions in general and TAVR in particular. There remains the need for a robust, consistent, clinically effective and cost effective solution for the closure of this (and other) large bore percutaneous access incisions so that such complications can be reduced to a minimum. This same issue exists for all large bore catheterization and cannulation devices and apparatus. Helping resolve this issue will ensure that patients are ambulated at the earliest possible time and their overall hospital stay is minimized as much as possible.

For the vast majority of transcatheter procedures that require access to the central vascular system (i.e. the Ascending Aorta, the Vena Cava, etc.), percutaneous access is typically obtained through an incision in the patient’s groin into either the Common Femoral Artery or the Femoral Vein. Through this access point, the required tools (e.g. guidewires, Introducers, Guide Catheters, Therapeutic devices etc.) are exchanged. At the end of the procedure, this access point must be hemostatically sealed before the patient can be ambulated. At present, all percutaneous closures of this access site are achieved using either:
A surgical cut-down method and the application of sutures directly to the arterial wall.
The use of a dedicated closure device that is delivered percutaneously via the same location of the original incision that was made in the first place.

Dedicated closure devices are by no means 100% effective and come with a variety of risks and procedural complications. Persistent bleeding around the delivered closure device is a common failing and requires the physician to apply external compression for a number of minutes before final completion of the overall procedure. Localized haematoma can also occur even after initial haemostatic closure appears to have been achieved which will require further intervention and potentially a return to the OR (operating room). The required use of large-bore introducers (typically in the range of 12Fr to 24Fr) for the primary therapeutic procedure often results in considerable trauma at the direct vessel point. Such trauma can lead to a structural weakness in the vessel at this point and may result in a pseudoaneurysm at this location at some point in the future. The use of substantial amounts of collagen in the construction of these closure devices (e.g. collagen plugs) can also have long term implications for the patient as it can lead to increased post-healing fibrosis at the incision site. In certain cases, this may result in the use of this access site being excluded as an option for any subsequent procedures that require similar access into the central vasculature since the fibrosed tissue can lead to additional problems in the completion of such procedures.

### SUMMARY OF THE INVENTION

The present invention seeks to overcome one or more of the disadvantage of the prior art. Particular and preferred aspects of the invention are set out in the accompanying independent claims. Combinations of features from the dependent claims may be combined with features of the independent claims as desired and appropriate and not merely as explicitly set out in the claims.

The term “comprising” as used herein to specify the inclusion of components also includes embodiments in which no further components are present.

According to a first aspect, there is provided a system for sealing an opening in a wall of a vessel defining a fluid-containing lumen, the system comprising:
a. an elongate and expandable support sleeve; and
b. a sleeve delivery apparatus arranged to receive the support sleeve, and expansion means to in-use expand the support sleeve into engagement with the wall of the vessel.

The vessel can be a blood vessel. Suitable blood vessels include arteries and veins.

Examples of openings in vessel walls include incision, punctures, perforations, and dissections of the vessel, e.g. vascular punctures, vascular perforations, and vascular dissections. Unlike other vascular sealing systems, the present invention is able to effect a seal with all such openings, so long as (a) the support sleeve is of a suitable length for the opening, and (b) the vessel is of suitable diameter to allow passage of the system to the opening.

The sleeve delivery apparatus (also referred to herein as the “sleeve delivery system”) can be used for transluminal delivery of the support sleeve along the vessel such that a medial portion of the support sleeve is adjacent to the opening. In certain embodiments, the sleeve delivery apparatus additionally comprises at least one radiopaque marker positioned longitudinally adjacent the support sleeve. In certain embodiments, the sleeve delivery apparatus comprises two radiopaque markers, one positioned at a forwards end of the support sleeve, the other positioned at a rearwards end of the support sleeve. Thus, when positioning the sleeve delivery apparatus relative to an opening in the wall of a vessel, the position of the opening can be marked (for example, in the case of an introducer sheath inserted into common femoral artery via a percutaneous incision, the introducer sheath can be radiopaque), and the radiopaque markers of the sleeve delivery apparatus can be positioned one either side of the opening. The position of the support sleeve relative to the opening in the vessel can therefore be confirmed to be correct, and the support sleeve can be positioned covering the opening in the vessel.

In certain embodiments, the system is a sheathless system, i.e. an outer (or retaining) sheath is not positioned around the support sleeve to restrain it prior to its expansion. In other embodiments, the system additionally comprises a moveable sheath dimensioned to cover the support sleeve. The sheath can be moveable (i.e. moveable longitudinally along the sleeve delivery apparatus) between a first position in which the sheath covers the support sleeve and a second position in which the sheath does not cover the support sleeve. The system can additionally comprise actuation means to move the sheath from the first position to the second position, i.e. to remove the sheath from the support sleeve.

In certain embodiments, the support sleeve is a hollow cylinder.

The support sleeve can be comprised of a polymeric material. In certain embodiments, the support sleeve is comprised of a resorbable material, particularly a resorbable polymer. Suitable polymers include (but are not limited to): polydioxanone (PDO), poly (I-lactic acid) (PLLA), poly(lactic-co-glycolic acid) (PLGA), poly lactic acid (PLA), poly glycolic acid (PGA), polycaprolactone (PCL), poly-d-lactic acid (PDLA), polycyanoacrylates, polyanhydrides, and polypropylene fumarate. Other suitable polymers will be readily apparent to one of ordinary skill in the art.

A particularly preferred polymeric material for the support sleeve is polydioxanone (PDO), which is a hygroscopic polymer that resorbs by hydrolysis, resulting in degradation by-products such as glycoxylate that can be excreted in urine, or that can be metabolised via a citric acid cycle to form carbon dioxide and water.

In certain embodiments, the abluminal surface (the outer surface) of the support sleeve is textured. The provision of a textured surface can assist in bonding the support sleeve to the vessel wall. This is particularly the case where hemostatic bonding of the support sleeve to the vessel wall is achieved.

In certain embodiments, the support sleeve includes (i.e. comprises) a secondary material disposed on or engaged with the support sleeve abluminal surface to promote platelet activation and recruitment between the abluminal surface and the wall of the vessel. In certain embodiments, the support sleeve includes a secondary material disposed on or engaged with the support sleeve abluminal surface to adhere the abluminal surface to the vessel wall. Suitable agents include, but are not limited to, collagen, chitosan, alginate, silica, and tantalum. In certain embodiments, Type II collagen is disposed on the support sleeve abluminal surface. In other embodiments, Type I collagen is disposed on the support sleeve abluminal surface.

In such embodiments including a secondary material disposed on or engaged with the support sleeve, the secondary material is preferably uniformly distributed on the support sleeve abluminal surface. To achieve a uniform distribution of e.g. Type II collagen, the type II collagen is first dissolved into a liquid and then sprayed through an electrical plasma discharge to produce a uniform layer on the support sleeve abluminal surface, the type II collagen being covalently bonded to the underlying substrate.

The secondary material can be deposited on the support sleeve using plasma deposition, particularly cold plasma deposition. Suitable plasma deposition techniques are taught in e.g. O′Sullivan, D et al., Appl. Sci. 2020, 10, 6670, doi:10.3390/app10196670 and O′Sullivan, D et al., ACS Omega 2020 Sep 24;5(39):25069-25076, doi: 10.1021/acsomega.0c02073, PMID: 33043185. The use of plasma deposition, particularly cold plasma deposition, provides for covalent bonding between the support sleeve and the substance/material (e.g. collagen) being deposited on the surface. Plasma deposition can be done in a non-aqueous solution, which therefore means that the surface onto which deposition is taking place is not exposed to water. For resorbable materials such as resorbable polymers, this can be particularly advantageous since it means that hydrolysis (and degradation) of the support sleeve is not initiated.

The provision of a sheath over the support sleeve also helps prevent the secondary material from contacting fluid in the vessel (e.g. blood) until the sheath is removed. Thus, platelet activation and recruitment by the secondary material does not occur until the sheath is removed from the support sleeve.

In certain embodiments, the support sleeve is drug-eluting. In certain embodiments, the luminal surface (inner surface) of the support sleeve is drug-eluting. Suitable drugs include, but are not limited to, paclitaxel. Paclitaxel is particularly useful in reducing restenosis. Other drugs include Taxus-based anti-restenotic drugs, Limus-based anti-restenotic drugs, tissue-repair pharmacological agents, and anti-inflammatory agents.

The expansion means (also referred to herein as an “expander device”) can be configured to expand upon actuation thereof to laterally (i.e. outwardly) expand the support sleeve. Expansion means can include an inflatable sac, for example an inflatable balloon. In other embodiments, the expansion means can comprise an expandable lattice structure. Examples include expandable lattice electrode catheters (Barkagan M. et al., Circ Arrhythm Electrophysiol. 2019; 12:e007090, doi: 10.1161/CIRCEP.118.007090, PMID: 31707809). The expansion means can be arranged to expand upon actuation thereof to laterally expand the support sleeve. Thus, the system can additionally comprise an actuator for the expansion means. The actuator can be configured to effect expansion of the expansion means from an initial (or unexpanded) state to an expanded state (with the support sleeve in engagement with the wall of the vessel), and also to effect contraction from an expanded state to an unexpanded state. In the case of an inflatable sac, the actuator can be a fluid delivery apparatus which is in fluid flow communication with the inflatable sac.

The support sleeve can be configured such that when in it is in engagement with the wall of the vessel in the absence of the expansion means, the support sleeve does not exert radial force on the wall of the vessel. Thus, the support sleeve acts to mask and protect the damaged tissue of the wall of the vessel. This is in contrast to prior art stents which exert a radial force on the wall of the vessel and which can then result in further damage to the wall and in collapsed stents.

In certain embodiments, the support sleeve is positioned (received) about the expansion means (such as an inflatable sac or balloon), the support sleeve and the expansion means collectively being folded for transluminal delivery along the vessel. Thus, folding of both the expansion means and the support sleeve can help reduce the overall cross-sectional area of the system. In certain embodiments, the folded support sleeve and expansion means are wrapped by a removable sheath. As noted above, the sheath can be removable prior to expansion of the support sleeve, and the system can additionally comprise actuation means to remove the sheath. The provision of a sheath over the support sleeve maintains the low profile for transluminal delivery along the vessel.

The sheath covering (which is positioned over the pleated and wrapped expansion means (e.g. balloon)-support sleeve combination) serves two principal purposes:
1) It ensures that the sleeve (and any coating of e.g. collagen on its abluminal surface) does not come into contact with blood until it is at the deployment location; and
2) It maintains the wrapped expansion means (e.g. balloon)-support sleeve combination in a low-profile configuration which allows for smooth transition into the body and smooth trackability within the vasculature.

Typically, folded balloons are heat set in order to improve fold memory retention. However, in embodiments of the present invention comprising a sheath, the balloon does not need to be heat set since the sheath constrains the inflatable sac/balloon. This can simplify manufacturing of the system. In more detail, one of the advantages that the sheath provides is the avoidance of the requirement of subjecting the expansion means (e.g. balloon)-support sleeve combination to a heat-setting process step in order to ensure that it stays in a low profile (i.e. wrapped) configuration as it transitions to the deployment site. Typically, a balloon will be pleated, folded, wrapped and heat-set in order to ensure that it maintains a low profile configuration up until the time that it is deployed/inflated. The sheath of the present invention does the job of maintaining this low profile configuration without the need for heat-setting.

In certain embodiments, the system additionally comprises a guidewire. Thus, the guidewire can be arranged (i.e. positioned) to extend through the vessel to proximate the opening in the vessel wall. The guidewire can be radiopaque to assist in correct positioning relative to the opening in the vessel wall. The sleeve delivery apparatus can be configured to travel along the guidewire to the opening in the vessel wall. Guidewires are well known in the art and include the likes of e.g. von Hessling A, et al. J Neurolntervent Surg 2021;0:1-6, PMID: 33947767, doi:10.1136/neurintsurg-2021-017296.

In certain embodiments, the system comprises a rapid exchange (RX) catheter.

Also provided according to the present invention is a method of sealing an opening in a wall of a vessel defining a fluid-containing lumen, the method comprising the steps of:
(i) inserting a system according to the present invention in a vessel remote the opening in the wall of the vessel to be sealed;
(ii) moving the sleeve delivery apparatus and the support sleeve such that they are positioned adjacent the opening in the wall of the vessel; and
(iii) expanding the expansion means so as to expand the support sleeve into engagement with the wall of the vessel.

As noted above, large bore catheter and cannula openings in vessels, particularly in the common femoral artery and femoral vein, are particularly problematic. It can often result in considerable trauma at the direct vessel point. Such trauma can lead to a structural weakness in the vessel at this point and may result in a pseudoaneurysm at this location at some point in the future. The use of substantial amounts of collagen in the construction of these closure devices (e.g. collagen plugs) can also have long term implications for the patient as it can lead to increased post-healing fibrosis at the incision site. In certain cases, this may result in the use of this access site being excluded as an option for any subsequent procedures that require similar access into the central vasculature since the fibrosed tissue can lead to additional problems in the completion of such procedures.

By effecting sealing of an opening in a vessel wall by way of a support sleeve inside (in the lumen of) the vessel, the sealing can be significantly better than that achieved with other systems and devices, and can reduce or avoid the fibrosis issues and mechanical damage resulting from them.

Where the system comprises a sheath, the sheath can be removed prior to the step of expanding the expansion sleeve. This can be done after the sleeve delivery apparatus and support sleeve are positioned adjacent the opening in the wall of the vessel.

For example, where a catheter or cannula opening has been created in e.g. the common femoral artery, a system of the present invention can be inserted into the radial artery in the arm of a patient (for example adjacent the wrist), and the sleeve delivery apparatus and support sleeve moved through the vasculature of the patient to the site of the opening in the common femoral artery. With the support sleeve correctly located, any sheath can then be moved such that it does not cover the support sleeve. The expansion means can then be expanded (actuated) so as to expand the support sleeve into engagement with the wall of the vessel. The expansion means may then be left in the expanded position for a predetermined period of time whilst bonding of the support sleeve to the wall of the vessel takes place. For example, this may be at least 15 seconds, or at least 30 seconds. In certain embodiments, it is between 15 and 60 seconds, more preferably between 15 and 45 seconds, for example between 15 and 30 seconds. The expansion means is then contracted, and the sleeve delivery apparatus is removed from the patient via the vessel remote the opening in the wall of the vessel which has now been sealed.

Thus, the method can additionally comprise the steps of:
(iv) contracting the expansion means; and
(v) removing the sleeve delivery apparatus from the vessel remote the opening in the wall of the vessel.

In certain embodiments, the opening in the wall of the vessel is in the wall of the common femoral artery, or the femoral vein.

In certain embodiments, the vessel remote the opening in the wall of the vessel to be sealed is the radial artery. Other suitable vessels will be readily apparent to one of ordinary skill in the art.

The vessel remote the opening in the wall of the vessel to be sealed can be in a different vessel. The vessel remote the opening can be in a superficial vessel.

Thus, the insertion site for the system can be of a smaller size (for example, a smaller diameter) than the opening in the wall of the vessel to be sealed. Preferably, the insertion site for the system is 6Fr or smaller. This compares with large bore devices which may be significantly larger (e.g. 12Fr) whose openings into blood vessels are to be sealed. For example, the insertion site for the system can have a cross-sectional area less than two thirds that of the opening in the wall of the vessel to be sealed, for example less than half or less than one quarter. For example, the insertion site for the system can have a circumference less than two thirds that of the opening in the wall of the vessel to be sealed, for example less than half of the opening in the wall of the vessel to be sealed.

The features and optional features of the system described above apply equally to the method of the present invention, and vice versa.

In one aspect, the present disclosure provides a support sleeve deployed inside the body vessel at a point of access through the wall of that vessel for the purposes of sealing that vessel so that the fluid contained within the vessel does not flow under pressure through the access point and escape to the surrounding tissue. The sleeve also provides structural support to the vessel so that natural healing of the access point is facilitated. The support sleeve is mounted on a minimally invasive balloon catheter which is delivered transluminally to the access location and which itself is introduced into the central vasculature at another location, most typically into the radial artery. Deployment of the sleeve is facilitated through the inflation of the balloon which applies positive contact pressure directly to the access point in the wall of the vessel and immediately effects hemostasis at that location. The balloon is then deflated after a number of seconds leaving the sleeve in place and the balloon catheter is removed in the normal way through its own minimally invasive access point.

These and other features, aspects, and advantages of the present disclosure will be apparent from a reading of the following detailed description together with the accompanying drawings, which are briefly described below. The present disclosure includes any combination of two, three, four, or more features or elements set forth in this disclosure, regardless of whether such features or elements are expressly combined or otherwise recited in a specific embodiment description herein. This disclosure is intended to be read holistically such that any separable features or elements of the disclosure, in any of its aspects and embodiments, should be viewed as intended, namely to be combinable, unless the context of the disclosure clearly dictates otherwise.

It will be appreciated that the summary herein is provided merely for purposes of summarizing some example aspects so as to provide a basic understanding of the disclosure. As such, it will be appreciated that the above described example aspects are merely examples and should not be construed to narrow the scope or spirit of the disclosure in any way. It will be appreciated that the scope of the disclosure encompasses many potential aspects, some of which will be further described below, in addition to those herein summarized. Further, other aspects and advantages of such aspects disclosed herein will become apparent from the following detailed description taken in conjunction with the accompanying drawings which illustrate, by way of example, the principles of the described aspects.

### DETAILED DESCRIPTION OF THE INVENTION

The present disclosure now will be described more fully hereinafter with reference to the accompanying drawings, in which some, but not all aspects of the disclosure are shown. Indeed, the disclosure may be embodied in many different forms and should not be construed as limited to the aspects set forth herein; rather, these aspects are provided so that this disclosure will satisfy applicable legal requirements. Like numbers refer to like elements throughout.

### DRAWINGS

FIG. 1 shows a radial artery access site for the Support Sleeve Delivery System (<6Fr) in the right wrist of a patient;

FIG. 2 is a partial cut-away view of the support sleeve delivery apparatus of FIG. 1;

FIG. 3 is a perspective view of a partial cut-away of the support sleeve delivery apparatus, the inflated balloon pressing the sleeve against the vessel wall;

FIG. 4 is a partial cut-away side view of the support sleeve delivery apparatus of FIG. 3;

FIG. 5 is a partial cut-away view of the sleeve which remains in place after the balloon has been deflated and the delivery system removed; and

FIG. 6 is a bar chart showing adhesion of PDO strips to fresh tissue, the PDO strips having varying thicknesses of collagen coating applied to their surface. Numerical values under the X-axis are the measured force (N).

### SEALING SLEEVE EMBODIMENT

One aspect/embodiment of the present disclosure is directed to providing an apparatus and method to effectively seal the large bore access point that is used to gain entry into the central vasculature; that large bore access point being used to facilitate the exchange of tools used during a large bore percutaneous transcatheter procedure.

### Design Solution

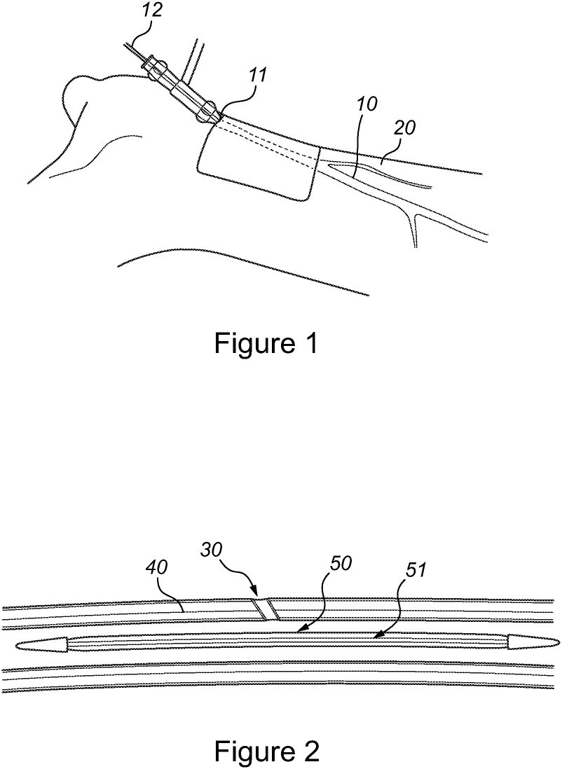

The design solution of this invention is to deliver a cylindrical support sleeve to the site of the access point in the lumen wall using an inflatable balloon catheter (or a self-expanding stent-type structure which is permanently attached to its delivery system and removed once the sleeve has been deployed). The sleeve is sized appropriately for the lumen diameter and the incision size/length. The delivery system used to deliver the support sleeve enters the patient’s body using a minimally invasive access point at some other location in the patient’s body. The delivery system travels over a guidewire which facilitates smooth transluminal delivery of the device to the correct deployment location. For example, the delivery system might enter the body through a minimally invasive access point in the radial artery of the patient’s wrist (FIG. 1) and transluminally traverse over a guidewire to the large bore incision that was made in the femoral artery for the purposes of facilitating tool exchange during a TAVR procedure. The delivery system (with sleeve) will remain in an undeployed low-profile state until it reaches the appropriate location for deployment. This positioning will typically be guided by some method of external vision system (e.g. ultrasound guidance or X-Ray fluoroscopy). The undeployed sleeve may be contained within a retractable sheath/covering in order to maintain a low-profile as it travels to the correct location and to ensure it does not contact blood until it is ready to be deployed.

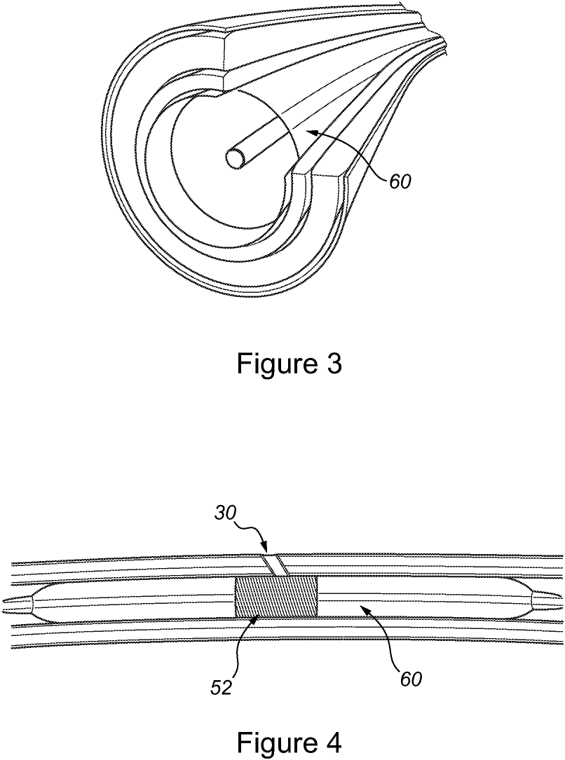

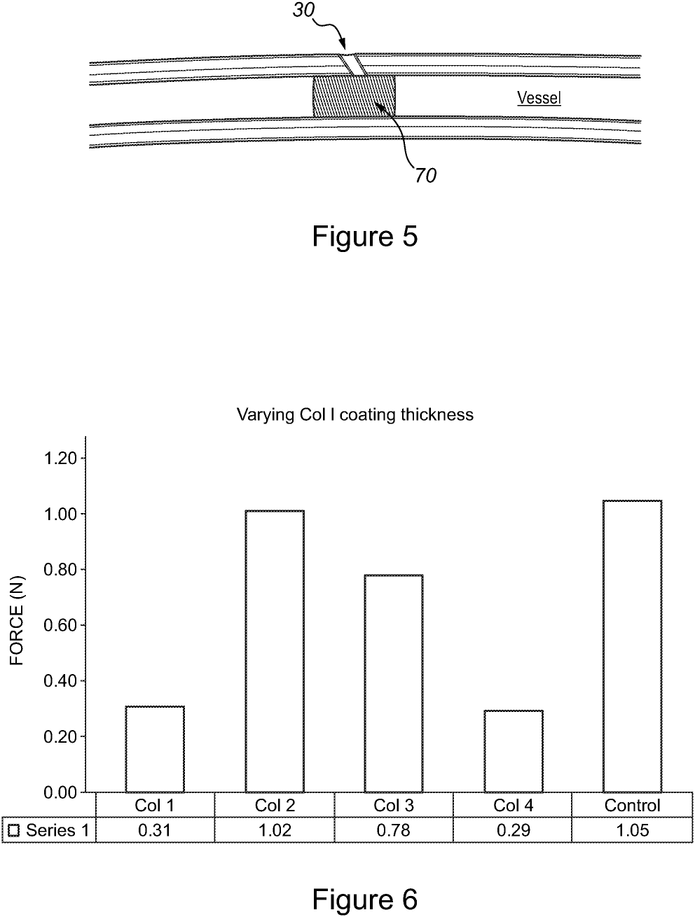

Once at the correct location (FIG. 2), the sleeve will be deployed through the inflation of the balloon or the expansion of a self-expanding stent-type structure. This will cause the sleeve to be pressed against the inner wall of the vessel and immediately seal the large bore incision (FIG. 3, FIG. 4). In the case of a blood-containing lumen, upon deployment the sleeve will initiate the blood coagulation cascade on the abluminal side of the sleeve. The resulting thrombus will act to adhere the sleeve to the inner wall of the lumen and result in instant hemostasis at the large bore incision point. The sleeve may contain some additional thrombus-inducing mechanisms on its abluminal surface such as a surface texturing (to increase the real surface area of the micro-structure) or the use of an additional material that will induce increased platelet activation and recruitment. Upon deflation of the balloon (or retraction of the self-expanding stent-like structure), the fluid pressure in the vessel will act in concert with the adhesion that has been enacted between the abluminal surface of the sleeve and the lumen wall itself. This dual mechanism ensures that hemostasis persists after the balloon is deflated and the sleeve remains in place as the delivery system is removed (FIG. 5). The delivery guidewire can remain in place for the entirety of the closure procedure until the physician is confident that hemostasis has been effected and large bore closure is complete.

The delivery system is removed from the patient’s body via its own minimally invasive incision point (e.g. the access point in the radial artery) which, upon final removal of the delivery guidewire and access Introducer sheath, can be closed through a simple external compression device (since this access incision will be typically be in the region of 5Fr - 6 Fr).

### Procedure

The procedure for using the above design is described below and is illustrated in FIGS. 1 - 5.

Step 1: Obtain access into the radial artery 10 using standard procedures and position a 6Fr Introducer sheath 11 into the artery 10 in the arm 20 of a patient to facilitate tool exchange into this artery.

Step 2: Insert a guidewire 12 (0.014″ (0.3556 mm) or 0.018″ (0.4572 mm)) into the radial artery 10 and advance it to the location of the large-bore incision site 30 to be sealed/closed in blood vessel 40.

Step 3: Insert the delivery system 50 containing the support sleeve 52 over the guidewire 12 and advance it to the location of the large-bore incision site 30 in blood vessel 40 to be sealed/closed (FIG. 2: wrapped balloon-sleeve combination 51 at the incision site 30 pre-deployment). Position correctly (using appropriate imaging methodologies) so that the support sleeve 52 is centrally positioned across the location of the large-bore incision site 30 to be sealed/closed.

Step 4: If the embodiment contains a protective sheath/covering over the sleeve-balloon combination 51, retract the protective sheath/covering which covers the undeployed sleeve thereby exposing the wrapped sleeve-balloon combination.

Step 5: The balloon 60 is inflated, thereby deploying the sleeve 52 and firmly pressing it against the inner wall of the lumen of the blood vessel 40.

Step 6: The physician will observe an immediate cessation of pulsatile flow at the skin level of the large bore access site.

Step 7: After a short number of seconds, the balloon can be deflated again leaving the support sleeve 52 in position. Hemostasis will persist- the insertion site 30 is sealed.

Step 8: Delivery system and delivery guidewire 12 are removed via the minimally invasive access location in the wrist. Closure of this minimally invasive access location can be effected through a simple external compression device.

### Advantages

Advantages associated with this closure support sleeve and the above-described procedure are significant and include the following:

1. Instant hemostatic closure through the use of a full cylindrical support sleeve delivered directly to the incision in the lumen wall.

2. The method of delivery through the inflation of a balloon applies focused pressure directly at the location where the incision in the lumen wall is. This means that compression of bleeding location is absolutely focused where it is most needed. This avoids the need for the physician to blindly apply compression over the access site through the skin of the patient which is time consuming, often not directed at the location required and requires physical effort on the part of the physician/nurse.

3. The cylindrical sleeve provides structural support to the injured vessel and helps with the healing process since it instantly takes pressure off the injury site once deployed.

4. The risk of a pseudoaneurysm forming around the incision is greatly reduced since the incision site (and the vessel itself) is supported by the sleeve. This protects the weakened vessel during the healing process.

5. The risk of a local hematoma forming around the incision is greatly reduced since the sealing ability provided by the sleeve is optimized due to the dual action of the adhesive effect provided by the coagulating blood between sleeve and vessel wall and the action of the vessel pressure acting to press the sleeve against the vessel wall post-delivery.

6. Given that the sleeve is bioresorbable, there will be no artefact left at the incision site after resorption has been completed. Furthermore, there will be no foreign material left in the tissue tract (i.e. the large-bore tract through which the TAVR tools were exchanged during the primary procedure) and so there will be no fibrosed tissue local to the incision site post-healing. This is important since it means that this location is not lost to that patient as an access point for future large-bore interventions as the tissue will heal close to its original integrity.

### Collagen Coating of Polydioxanone (PDO) Strips

This experiment was undertaken in order to determine the adhesion (bonding) between PDO coated with collagen and fresh tissue.

PDO strips were coated with Type I collagen using cold plasma deposition and pressed against fresh tissue in the presence of blood. The resulting test pairs were then subjected to tensile testing in order to determine the adhesive force between the PDO coupon and the tissue can be measured. A surgical glue was used as the control in place of the Type I collagen.

As illustrated in FIG. 6, the varying thicknesses of collagen achieved excellent results in terms of adhesion of the PDO strips to fresh tissue. This demonstrates that the use of a secondary material to promote platelet activation and recruitment can result in excellent hemostatic adhesion (bonding) of the support sleeve to the wall of the vessel.

Many modifications and other embodiments of the inventions set forth herein will come to mind to one skilled in the art to which these disclosed embodiments pertain having the benefit of the teachings presented in the foregoing descriptions and the associated drawings. Therefore, it is to be understood that embodiments of the invention are not to be limited to the specific embodiments disclosed and that modifications and other embodiments are intended to be included within the scope of the invention. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the disclosure. In this regard, for example, different combinations of elements and/or functions than those explicitly described above are also contemplated within the scope of the disclosure. Although specific terms are employed herein, they are used in a generic and descriptive sense only and not for purposes of limitation.

It should be understood that although the terms first, second, etc. may be used herein to describe various steps or calculations, these steps or calculations should not be limited by these terms. These terms are only used to distinguish one operation or calculation from another. For example, a first calculation may be termed a second calculation, and, similarly, a second step may be termed a first step, without departing from the scope of this disclosure. As used herein, the term “and/or” and the “/” symbol includes any and all combinations of one or more of the associated listed items.

As used herein, the singular forms “a”, “an” and “the” are intended to include the plural forms as well, unless the context clearly indicates otherwise. It will be further understood that the terms “comprises”, “comprising”, “includes”, and/or “including”, when used herein, specify the presence of stated features, integers, steps, operations, elements, and/or components, but do not preclude the presence or addition of one or more other features, integers, steps, operations, elements, components, and/or groups thereof. Therefore, the terminology used herein is for the purpose of describing particular embodiments only and is not intended to be limiting.

Other aspects of the invention include the following:

Aspect 1. A system for sealing an incision in a wall of a vessel defining a fluid-containing lumen, comprising:
a. an elongate and expandable support sleeve; and
b. a sleeve delivery system arranged to receive the support sleeve and to transluminally deliver said support sleeve along the vessel such that a medial portion of the support sleeve is adjacent to the incision, the sleeve delivery system being further arranged to laterally expand the support sleeve into engagement with the wall of the vessel about the incision so as to seal the incision.

Aspect 2. The system of Aspect 1, wherein the support sleeve is a hollow cylinder.

Aspect 3. The system of Aspect 1, wherein the support sleeve is comprised of a polymeric material.

Aspect 4. The system of Aspect 1, wherein the support sleeve is comprised of a resorbable material.

Aspect 5. The system of Aspect 1, wherein the support sleeve includes an outer abluminal surface, and wherein the abluminal surface is textured.

Aspect 6. The system of Aspect 1, wherein the support sleeve includes an outer abluminal surface, and wherein the support sleeve includes a secondary material disposed on or engaged with the abluminal surface to promote platelet activation and recruitment between the abluminal surface and the wall.

Aspect 7. The system of Aspect 1, wherein the support sleeve includes an outer abluminal surface, and wherein the support sleeve includes a secondary material disposed on or engaged with the abluminal surface to adhere the abluminal surface to the wall.

Aspect 8. The system of Aspect 1, where the sleeve delivery system is arranged to receive the support sleeve into engagement with an expander device, and wherein the expander device comprises an inflatable sac arranged to expand upon actuation thereof to laterally expand the support sleeve.

Aspect 9. The system of Aspect 1, where the sleeve delivery system is arranged to receive the support sleeve into engagement with an expander device, and wherein the expander device comprises an expandable lattice structure arranged to expand upon actuation thereof to laterally expand the support sleeve.

Aspect 10. The system of Aspect 1, where the sleeve delivery system is arranged to receive the support sleeve about a guide wire, and wherein the guide wire is arranged to extend through the vessel to proximate the incision such that the support sleeve is directed to the incision along the guide wire.

Aspect 11. The system of Aspect 1 where the sleeve delivery system includes an expander device, wherein the expander device comprises an inflatable sac, and wherein the support sleeve is received about the inflatable sac with the support sleeve and the inflatable sac collectively being folded to achieve a low profile for transluminal delivery along the vessel.

Aspect 12. The system of Aspect 11 where the folded support sleeve and inflatable sac are wrapped by a sheath to maintain the low profile for transluminal delivery along the vessel, the sheath being removable prior to expansion of the support sleeve.

### REFERENCE SIGNS

10 - radial artery
11 - introducer sheath
12 - guide wire
20 - arm
30 - Incision site / tissue tract
40 - blood vessel
50 - delivery system
51 - wrapped balloon-sleeve combination at the incision site pre-deployment
52 - support sleeve
60 - balloon pressing the sleeve against the vessel wall.
70 - support sleeve (across large-bore incision 30)

1. A system for sealing an opening in a wall of a vessel defining a fluid-containing lumen, the system comprising:
a. an elongate and expandable support sleeve; and
b. a sleeve delivery apparatus arranged to receive the support sleeve, expansion means to in-use expand the support sleeve into engagement with the wall of the vessel.

2. A system according to claim 1, wherein the vessel is a blood vessel.

3. A system according to claim 1, wherein the sleeve delivery apparatus is a sheathless sleeve delivery apparatus.

4. A system according to claim 1, wherein the sleeve delivery apparatus additionally comprises a moveable sheath dimensioned to cover the support sleeve, the sheath being moveable between a first position in which the sheath covers the support sleeve and a second position in which the sheath does not cover the support sleeve.

5. The system of claim 1, wherein the support sleeve is a hollow cylinder.

6. The system of claim 1, wherein the support sleeve is comprised of a polymeric material.

7. The system of claim 1, wherein the support sleeve is comprised of a resorbable material.

8. The system of claim 1, wherein the support sleeve includes an outer abluminal surface, and wherein the abluminal surface is textured.

9. The system of claim 1, wherein the support sleeve includes an outer abluminal surface, and wherein the support sleeve includes a secondary material disposed on or engaged with the abluminal surface to promote platelet activation and recruitment between the abluminal surface and the wall of the vessel.

10. The system of claim 1, wherein the support sleeve includes an outer abluminal surface, and wherein the support sleeve includes a secondary material disposed on or engaged with the abluminal surface to adhere the abluminal surface to the vessel wall.

11. The system of claim 1, wherein the expansion means comprises an inflatable sac.

12. The system of claim 11, wherein the expansion means comprises an inflatable sac, and wherein the support sleeve is positioned about the inflatable sac, the support sleeve and the inflatable sac collectively being folded for transluminal delivery along the vessel.

13. The system of claim 12, wherein the folded support sleeve and inflatable sac are wrapped by a removable sheath.

14. The system of claim 1, wherein the expansion means comprises an expandable lattice structure.

15. The system of claim 1, additionally comprising a guidewire.

16. A method of sealing an opening in a wall of a vessel defining a fluid-containing lumen, the method comprising the steps of:
(i) inserting a system according to claim 1 at an insertion site in a vessel remote the opening in the wall of the vessel to be sealed;
(ii) moving the sleeve delivery apparatus and the support sleeve such that they are positioned adjacent the opening in the wall of the vessel; and
(iii) expanding the expansion means so as to expand the support sleeve into engagement with the wall of the vessel.

17. A method according to claim 16, additionally comprising the steps of:
(iv) contracting the expansion means; and
(v) removing the sleeve delivery apparatus from the vessel remote the opening in the wall of the vessel.

18. A method according to claim 16, wherein the insertion site for the system is of a smaller size than the opening in the wall of the vessel to be sealed.
